(19) United States
(12) Patent Application Publication
(10) Pub. No.: US 2005/0054907 A1
(43) Pub. Date: Mar. 10, 2005

(54) Highly portable and wearable blood analyte measurement system

(76) Inventors: Joseph Page, La Jolla, CA (US); James Plante, Del Mar, CA (US)

Correspondence Address: Joseph Page, PO Box 757, La Jolla, CA, 92038, US

(21) Appl. No.: 10/656,376

(22) Filed: Sep. 8, 2003

(51) Int. Cl. A61B 5/00

(52) U.S. Cl. 600/316; 600/344

(57) ABSTRACT

Non-invasive wearable systems for continuous measurement of blood glucose concentrations help diabetics maintain best awareness and control. A wearable article such as a wristwatch includes elements integrated therewith to provide for biometric measurements. Specifically, both optical and acoustic transducers are arranged within an article such that they are coupled to tissue in a manner which permits blood analytes measurements to be made. In best versions, a quantum cascade laser is arranged with crystalline acoustic detectors in a photoacoustic effect measurement scheme. Laser pulses stimulate special vibrational states of glucose molecules to produce an acoustic return signal to be received at a piezoelectric detector. A wristwatch case may include a back member which supports arrangements and coupling between the back of the watch, elements contained therein, and tissue in contact with the device.

### BACKGROUND OF THE INVENTIONS

1. Field

The following invention disclosure is generally concerned with: biometric instruments arranged to be worn and specifically concerned with: non-invasive blood glucose measurement systems integrated with common wearable apparatus.

2. Prior Art

Systems have been formed to provide biometric measurements in conjunction with being worn on the body. For example, blood pressure measurement systems may be worn about the upper arm. A chamber is inflated and interacts with the body. A display presents a user with an indication of the blood pressure.

It is possible to monitor and measure body temperature with a thermometer which may be coupled to the body and worn thereabout. In appropriate thermal contact, an electronic thermocouple might provide a conversion from temperature to electronic signal which can be displayed at a monitor or display readout.

Heart rate monitors have been arranged so they might be worn while they constantly measure a user's pulse or heart rate. A chest strap might secure a transducer to the chest where a strong signal indicative of heart beat is coupled to an audio pick-up. The signal may be passed to a display integrated with a monitor which may be worn about the wrist.

A very special class of heart rate monitors is incorporated completely within a wristwatch case. Optical elements such as light emitting diodes are used to illuminate flesh from the backside of the watchcase. Special lenses are used to recover light after it has interacted with flesh. The signal may contain information relating to pulse, and implicitly heart rate. Further, display of heart rate information may be made at the front of the watch case.

An important wearable instrument has been designed to take blood glucose measurements. The instrument commercially known as ‘GlucoWatch’ includes a tissue interface at the backside of the watch that is in contact with a user's skin. Under the influence of electrical currents, interstitial fluids containing glucose molecules are drawn to the device. Received therein, the glucose molecules are chemically analyzed in processes internal to the apparatus.

Accordingly, it is certainly true that one could say biometric measurement systems have been arranged as wearable devices. More specifically, these devices might be integrated with articles commonly worn such as a wrist watch.

Another important body of the prior art also contains various optical measurement systems for determining concentrations of blood analytes and/or including blood glucose concentrations for diabetics. These include: near-IR, all-optical spectroscopy systems; near-IR photoacoustic measurement systems; mid-IR photoacoustic systems; among others.

U.S. Pat. No. 4,169,676 to Kaiser, shows methods of glucose measurement by putting a sensor directly against the skin or against the tongue. The procedure and device shown there uses a laser and determines the content of glucose in a specific living tissue sample by comparing the IR absorption of the measured material against the absorption of IR in a control solution by use of a reference prism.

Dahne et al., teach in U.S. Pat. No. 4,655,255, an apparatus for non-invasively measuring the level of glucose in a blood stream or tissues. These methods are photometric and use light in the infrared spectral region. These procedures use light in the 1.0 to 2.5 micron range. Dahne's device is jointly made up to two main sections, a light source and a detector section situated about a body part such as a finger. Infrared light is achieved by use of filters placed after a broadband source. The detector section is made up of a light-collecting integrating sphere or half-sphere leading to a means for detecting wavelengths in the near-infrared region. Dahne et al. goes to some lengths teaching away the use of IR light having wavelengths greater than about 2.5 microns since those wavelengths are strongly absorbed by water and have very little penetration capability into living tissues containing glucose.

Rosenthal et al., describes a non-invasive glucose monitoring device using near-IR light in their U.S. Pat. No. 5,028,787. Light is passed into the body in such a way that it passes through some blood-containing region. The so-transmitted or reflected light is then detected using an optical detector. The near-IR light sources are preferably infrared emitting diodes (IRED).

Harjumaa et al, teaches in U.S. Pat. No. 5,178,142, to use a stabilized near-IR radiation beam containing two alternating wavelengths in a device to determine a concentration of glucose or other constituents in a human or animal body. The amplitude of the varying alternating signal is detected and is said to represent glucose concentration or is taken to represent the difference in glucose concentration from a preset reference concentration.

U.S. Pat. Nos. 5,179,951 and 5,115,133, to Knudson, show application of IR light for measuring blood glucose levels directly in blood vessels in the tympanic membrane. Detected signals are amplified, decoded, and, using a microprocessor, provided to a display device. The IR detector includes “means for detecting the temperature of the volume in the ear between the detector and the ear's tympanic membrane.”

In U.S. Pat. No. 5,433,197, Stark describes a non-invasive glucose sensor. IR radiation is passed into the eye through the cornea and the aqueous humor, reflected from the iris or the lens surface, and then passed out through the aqueous humor and cornea. Reflected radiation is collected and detected by an IR sensor which measures the reflected energy in one or more specific wavelength bands. Comparison of reflected energy with source energy provides a measure of the spectral absorption by the eye components. Measured glucose concentration in the aqueous humor tracks that of the blood by a fairly short time. The infrared source is an LED with a refraction grating so that the light of a narrow wavelength band 10 to 20 nanometers wide passes through the exit slit. Use of IR spectrum below 1.4 microns and in the region between 1.5 and 1.8 microns is suggested.

U.S. Pat. No. 5,267,152, to Yang et al., shows a non-invasive method and device for measuring glucose concentration. Near-IR radiation, specifically with a wavelength of 1.3 microns to 1.8 microns from a semiconductor diode laser is used as an optical source. Light is transmitted down through the skin to the blood vessel where light interacts with various components of the blood and is then diffusively reflected by the blood back through the skin for measurement of the resulting spectrum.

Inventor Kuperschmidt presents a device in U.S. Pat. No. 5,398,681, which is said to be a pocket-type apparatus for measurement of blood glucose using polarization techniques. Glucose tends to rotate the polarization of light passing therethrough. Laser light is introduced into a finger or ear lobe and the phase difference between a reference signal and the measurement signal is measured and processed to formulate and calculate a blood glucose concentration which is then displayed.

U.S. Pat. No. 6,001,067 shows an implantable device suitable for glucose monitoring. It utilizes a membrane in contact with a thin electrolyte phase, which in turn is covered by an enzyme-containing membrane, e.g., glucose oxidase in a polymer system. Sensors are positioned in such a way that they measure the electro-chemical reaction of the glucose within the membranes. That information is then passed to desired sources.

Marchitto et al present yet another system for biological measurement which is minimally invasive as U.S. Pat. No. 6,387,059. In this teaching, pulsed light is used to form a microblister and draw interstitial fluid to the surface of the skin where it can be collected for chemical analysis. This technique, while using optical pulsed energy to draw a reaction from the tissue, does not directly measure the optical response in the tissue as a results of absorption or optical scatter.

A special class of non-invasive in vivo optical glucose measurement includes one based upon phenomena known as a ‘photoacoustic effect’ PA. The following patents relate primarily to systems employing photoacoustic effects.

U.S. Pat. No. 5,657,754 by inventor Rosencwaig primarily describes apparatus for the non-invasive analysis of non-homogeneous samples. The apparatus is suited for analyzing biological samples. An intensity modulated light beam is used to preferentially heat a selected constituent in the sample. Such periodic heating causes thermal waves in the test medium.

Inventor Chou of California teaches a method and apparatus for non-invasive measurement of blood glucose by photoacoustics in U.S. Pat. No. 5,941,821 published Aug. 24, 1999. Heating at the surface of a tissue causes surrounding air to also heat and produce a response signal which is measured or detected via a differential microphone. Further, optical energy is delivered to the tissue via a fiber optic coupling element. The same inventor further presents in a similar disclosure, U.S. Pat. No. 6,049,728 additional detail relating to these techniques.

Oraevsky and others teach special real time optoacoustic monitoring of changes in tissue properties via important optoacoustic imaging technique. U.S. Pat. No. 6,309,352 dated Oct. 10, 2001. These systems, while being directed to monitor tissues, are not suitable for blood analyte measurement but rather are aligned with the task of control during operational procedures occurring simultaneously with the measurement. Oraevsky additionally presents an interesting technique of analysis with regard to the spatial profile of an optically-induced acoustic transient. This recent disclosure is U.S. Pat. No. 6,405,069 dated Jun. 11, 2002. In some versions, the technique includes addressing tissue via the eye which has better access to certain components not readily available in techniques addressing tissue via dermal layers.

U.S. Pat. No. 6,466,806 of Oct. 15, 2002 presents very special technique characterized as resonant photoacoustic spectroscopy. This involves tuning optical pulses to cause a resonant acoustic wave. In accordance with a reference database, the parameters of the pulses which cause resonance suggests features of the material such as blood analytes concentration.

U.S. Pat. No. 6,526,298 by inventors Khalil et al, describes techniques with emphasis in temperature compensation. The skin sometimes is highly variable in temperature and tends to make difficulties in some measurement configurations. Accordingly, steps may be taken to reduce or compensate for temperature variances as taught by these researchers.

Finally, U.S. Pat. No. 6,484,044 by Lilienfeld-Toal presents a very useful technique for determining blood glucose concentration via a photoacoustic effect process. However, this system is restricted to single wavelength embodiments which tend to want accuracy. Further, the device is large in that its optical sources and detectors require significant support apparatus. The system might preferably be designed as a ‘table-top’ instrument.

While systems and inventions of the art are designed to achieve particular goals and objectives, some of those being no less than remarkable, these inventions have limitations. Inventions of the art are not used and cannot be used to realize the advantages and objectives of these inventions taught in this presentation and disclosure.

Important details relating to biometric measurement systems using Mid-IR photoacoustic effect are found in disclosures submitted to the USPTO by these same inventors in September 2003 entitled: “Mid-IR Non-Invasive Blood Analyte Measurement Systems” and another entitled: “Spatial Detectors for In-vivo Measurement of Glucose Concentration”. These entire disclosures are hereby incorporated into this disclosure by reference. It should be understood that all of the herein referenced materials provide considerable definitions of elements of these inventions. The instant specification may rely upon those disclosures for enablement of the particular teachings of each.

### SUMMARY OF THESE INVENTIONS

Comes now, Joseph Page and James Plante with inventions of portable, wearable blood analyte measurement systems including miniature devices integrated within common articles such as a wristwatch.

A highly unique combination of elements forms a photoacoustic effect system operable for making blood analyte measurements. The elements from which these systems are comprised are highly miniaturized and tightly integrated to fit within the very tight space and power constraints compatible with articles which may be worn on the body and further be in intimate contact therewith. In one best mode, a blood glucose concentration detector is integrated with a common wristwatch. The backside of the wristwatch is specially prepared and configured to support the necessary transducers of a photoacoustic system; i.e. an optical source and acoustic detector. In preferred versions, a quantum cascade laser emits radiation at Mid-IR wavelengths. This light is properly coupled into tissue where glucose molecules may be found. A well designed acoustic detector, i.e. a PZT crystal also integrated with the backside of the wristwatch, is similarly coupled to the tissue comprised of glucose molecules. Both the laser and detector are well in contact with the skin via the wristwatch case to form a complete photoacoustic effect measurement system. When the device is worn at the wrist like any common watch, the system transducers form a critical coupling to the precise layer of skin necessary for reliable blood analyte measurement. As such, a user need not manipulate or activate the system in any way while the system remains well disposed to make continuous and accurate measurements.

It is advantageous to combine an advanced instrument with an article like a wristwatch because it encourages better and more complete use of the instrument. Competing devices may include tabletop units or portable units such as those which may be carried in a handbag. These devices are less likely to be used as they necessarily require being handled to acquire a reading and that takes the direct attention of the user which tends to interrupt a normal course of activity. Conversely, a device worn continuously may make frequent measurements without taking any conscious effort from the patient. In this regard, the device which can be worn is more effective than those portable devices not wearable.

It is a primary function of these systems to provide greatly improved blood glucose monitoring. It is a contrast to prior art methods and devices that systems of the art do not permit continuous and comfortable measurement of blood glucose concentration. A fundamental difference between devices of the instant inventions and those of the art can be found when considering its integration within a wearable apparatus such as a wristwatch.

### Objectives Of These Inventions

It is a primary object of these inventions to provide wearable biometric devices.

It is an object of these inventions to provide blood glucose monitoring systems integrated with articles which may be worn about the body.

It is a further object to provide non-invasive techniques of measuring blood glucose over long periods.

A better understanding can be had with reference to detailed description of preferred embodiments and with reference to appended drawings. Embodiments presented are particular ways to realize the invention and are not inclusive of all ways possible. Therefore, there may exist embodiments that do not deviate from the spirit and scope of this disclosure as set forth by the claims, but do not appear here as specific examples. It will be appreciated that a great plurality of alternative versions are possible.

### BRIEF DESCRIPTION OF THE DRAWING FIGURES

These and other features, aspects, and advantages of the present invention will become better understood with regard to the following description, appended claims and drawings where:

FIG. 1 is a sketch showing the relation between a wristwatch and a wearer;

FIG. 2 illustrates important elements of a wearable apparatus in the form of a wristwatch;

FIG. 3 presents a face view of a wristwatch;

FIG. 4 is a side view of a similar wristwatch emphasizing the device package;

FIG. 5 includes a block diagram of major watch elements which may be incorporated within a package such as a watch case;

FIG. 6 shows a device of these inventions in relation with tissue of a user.

FIG. 7 illustrates a user interface display operable for presenting data in a user friendly format.

### GLOSSARY OF SPECIAL TERMS

Throughout this disclosure, reference is made to terms which may or may not be exactly defined in popular dictionaries as they are defined here. To provide a more precise disclosure, the following terms are presented with a view to clarity so that the true breadth and scope may be more readily appreciated. Although every attempt is made to be precise and thorough, it is a necessary condition that not all meanings associated with each term can be completely set forth. Accordingly, each term is intended to also include its common meaning which may be derived from general usage within the pertinent arts or by dictionary meaning. Where the presented definition is in conflict with a dictionary or arts definition, one must use the context of use and liberal discretion to arrive at an intended meaning. One will be well advised to error on the side of attaching broader meanings to terms used in order to fully appreciate the depth of the teaching and to understand all the intended variations.

A Wearable article—is worn at or on the body via some coupling means such as a belt or wristband. The article is in close proximity and may form intimate contact with skin lying just next to the article.

Photoacoustic Effect Spectroscopy—is the process by which test matter is illuminated with pulsed optical energy which is converted to waves of acoustic energy which may reflect the nature of the matter being probed.

Quantum Cascade Laser—is a special class of semiconductor laser which is highly efficient and may be miniaturized and incorporated as a subsystem of a more complex instrument.

Mid-IR—is a spectral range in the optical domain best described as between about 2 and 200 microns.

Case/Body—is a shell element formed to contain and support therein a plurality of subsystems some may be unrelated to others.

### PREFERRED EMBODIMENTS OF THESE INVENTIONS

In accordance with each of preferred embodiments of these inventions, there is provided blood glucose concentration measurement apparatus. It will be appreciated that each of embodiments described include an apparatus and that the apparatus of one preferred embodiment may be different than the apparatus of another embodiment.

[intro . . . ]

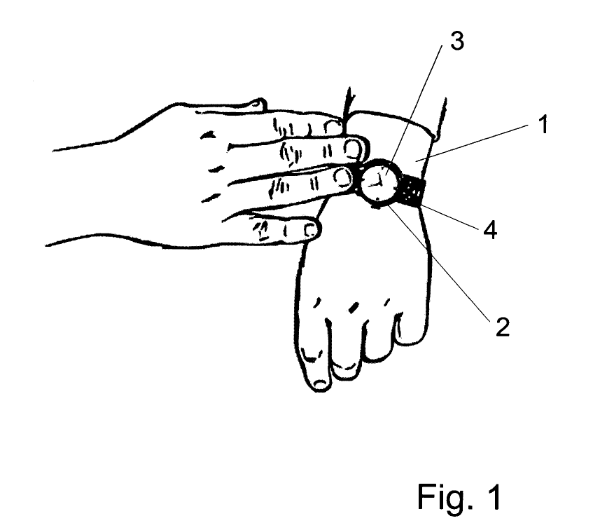

Preferred embodiments of these inventions may be more readily and fully appreciated in view of the following discussion with reference to the drawing figures. FIG. 1 illustrates a user wearing thereon his wrist 1, a watch 2 type instrument. The watch face 3 provides a user interface where information relating to date and time is presented to its user. The apparatus is worn snugly about the wrist by way of wrist band 4. One will recall the backside of the watch body is in intimate contact with the skin at the top of the wearer's wrist. In normal use of wrist watches, the watch remains is good contact with its wearer without excessive tightening of the watch band. In cases where users prefers wearing a watch quite loosely, a step to hold the watch during a measurement might be relied upon to keep the watch stationary for a momentary probing operation. The drawing illustrates a user temporarily holding the watch during a prescribed period where a blood glucose concentration measurement is made. It is understood that proper operation might require brief periods of attention from a user/operator.

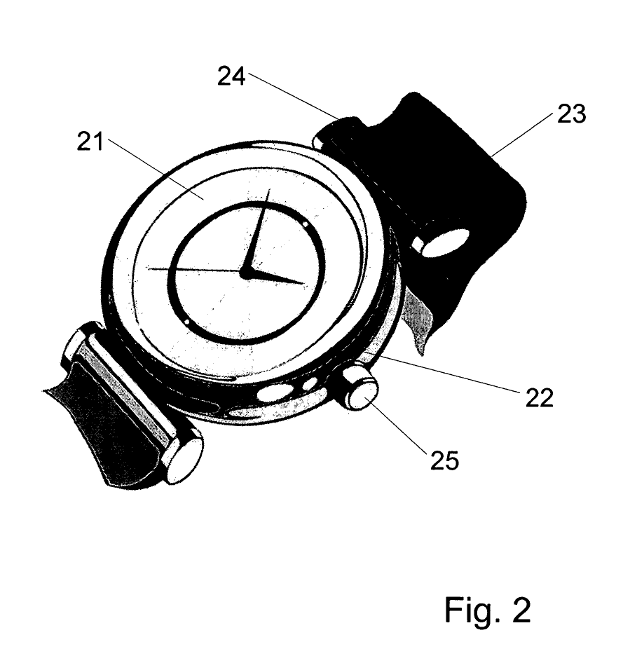

FIG. 2 shows a common wristwatch is better detail. A watch case is comprised of a topside or ‘face’ 21 which provides means to present information to a user. A transparent lens or crystal can provide necessary protection while simultaneously allowing viewing access to indicia interior to the case. Typically of course, this is merely the time of day. However, one can surely attest that some advanced watches are arranged to present date, calendar, position, altitude, heart rate, stopwatch, among many other functional information and figures. Thus the watch designed for wear about the human wrist includes a watch case formed of a rigid body made sometimes from metals or very durable plastics, or combinations thereof. The watch is secured circumferentially to a wrist via wrist band 23. The watch band is connected to the watch case via some linking means such as the pin 24 arrangement shown.

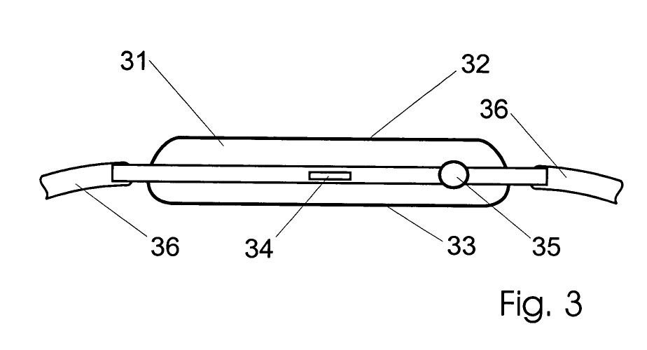

FIG. 3 illustrates a side view of a wrist watch having a slightly enlarged case 31. The topside 32 may include a transparent window which can be used as part of a user interface. Information developed or maintained in the device can be presented to a user at a display there. A bottom side 33 is prepared in a special manner to couple both optical and acoustic transducers to skin. The bottom side might include mounting systems appropriate to receive therein lenses, windows, or other elements. The watch case bottom side in these inventions is distinguish from a typical case bottom side in that those pieces are generally simple flat metallic sealing members. The watch may include user controls such as tactile knob 34 which can be manipulated easily with ones fingers to provide feedback to devices in the case interior. A data output port 35 is arranged to provide a communication mechanism whereby stored information within the case may be passed to outside sources such as a computer. While electrical connectors are inexpensive and convenient, for purposes of this disclosure it will be understood that a data output port might also include mechanisms such as IR communications links, so called Bluetooth radio systems, among other wireless techniques. So long as data is passed to and/or from, it is a ‘data output port’. A watch band 36 can be attached appropriately via a bracket to complete the ensemble.

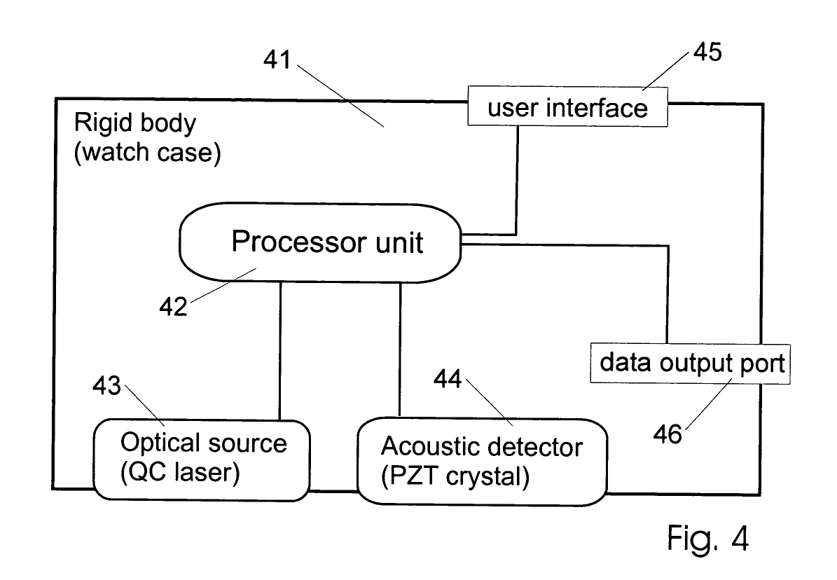

In special wrist watch cases, there is enough space to accommodate the following subsystems which are better defined with reference to the block diagram of FIG. 4. A rigid case 41 or body element houses and contains the primary elements from which a wearable biometric measuring device may be comprised. The case may form an enclosed space of between one and ten cubic centimeters such that the article may be easily worn about the body. The case has a top side and a bottom side. The top side is arranged to support user interface function and structure, while the bottom side support transmission of optical and acoustic signals from the interior of the case to the exterior by way of subsystems. A photoacoustic effect, blood glucose concentration measurement system may include a processing unit 42. A special purpose computing processor unit can be arranged to serve the task at hand with programming permanently formed within the device. The purpose build processor is arranged with a view to driving the optical source, receiving acoustic return signals, processing returned signals, referring to data table references, providing an output indicative of blood analyte levels, among others. Accordingly, the processor is in electronic communication with an optical source 43, acoustic detector 44, user interface 45 and data output port 46. The optical source, acoustic detector, data output port and user interface are drawn to overlap the periphery of the case to indicate that each of those elements may communicate with the exterior of the case in some manner. For example, the optical source may pass light beams through the case, via a window, into tissue.

In best versions, the optical source is at least one specialized semiconductor laser sometimes known as a ‘quantum cascade laser’, QC laser. The processor delivers laser driving pulses to activate the laser in accordance with a pre-selected modulation scheme. For photoacoustic systems, this includes a set of short pulses carefully spaced in time. In advanced versions, the optical source includes a plurality of lasers each at different frequency than another. In this way, a prescribed spectrum can be design with regard to the element being targeted which might have an optical response signature particular to that molecule.

QC lasers of these inventions are driven in a very special manner unlike common QC lasers. Because these devices are necessarily tightly integrated within a small package, there is no support for consumption of large quantities of energy. Thus, these devices are must be driven sparingly with timed measurements arranged over specified periods. In this way, power can be conserved such that the laser may be driven with the limited energy of a pill battery. Or example, in one scenario, a blood glucose measurement may be desirable each 30 minutes. Upon a trigger, the optical source is activated to produce a series of very short pulses separated it time by a prescribed amount. In this way, the duty cycle of the laser is very, very low and consumes only a small amount of energy. It remains very useful to have 48 blood glucose measurements through the course of a single day in most aggressive diabetes management plans.

The processor unit is in further communication with an acoustic detector, a transducer which converts pressure changes into an electronic signal. In some preferred versions, these are piezoelectric PZT crystals. Piezoelectric devices are solid state elements having small size, light weight. They are readily available and easily miniaturized. Signals generated at the PZT are converted into electronic pulses and returned to the processor unit for processing. Both the optical source and the acoustic detector are further integrated with the case bottom side whereby proper coupling to the target tissue is established when the case is worn at the wrist. Optional elements, ‘data output port’ 45 and ‘user interface’ 46 may be used as necessary in agreement with the objectives of various versions. A user interface permits measurement results to be displayed to a user, perhaps in real-time, for example at a watch face in the top side of the case.

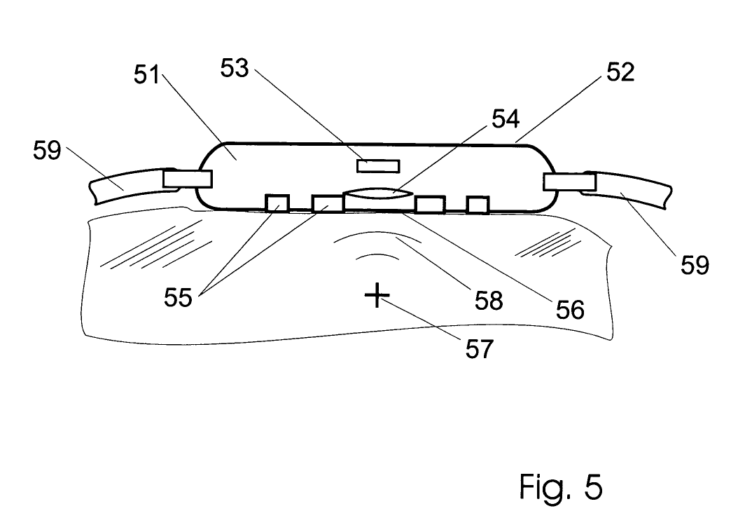

FIG. 5 is a side cut away view with certain elements shown in positional relation to others as they might be in use. The main body 51 forms a watch case of the wrist watch type. Said watch case may include a top side portion 52 to permit user interface via a display means under a transparent window. Quantum cascade lasers 53 produce optical pulses in wavelengths in agreement with natural resonances of glucose molecules (for example). Optical beams may be coupled to tissue via a lens 54 designed to focus and concentrate light. It is preferred to get light beams deep into the tissue past the uppermost epidermis layer of skin. A plurality of short pulses may be produced with a predefined period which supports use of a multi-element acoustic detector 55. Optical pulses may be transmitted through a special window 56 in the case bottom side. The bottom side of the case is formed with special features to support a window for optical coupling and spaces which permit acoustic coupling between tissue and the detector. A multi-element detector may be inset into spaces in a metallic support for example. It is very important to note that FIG. 5 is not drawn to scale. While the normal width of a common wrist watch is between about 10-30 centimeters, the depth in tissue of a good target point is typically on the order of 100 microns. Thus target point 57 shown below the surface of the tissue is grossly out of scale; it is drawn this way for clarity. Similarly, acoustic detector elements may also be quite small in comparison to how they are shown in the drawing. The drawing is not meant as an engineering or mechanical drawing. Pressure waves 58 emitted from the target point 57 propagate towards the watch body bottom side where they are received at the acoustic detector. The entire apparatus may be coupled via a frame to a watch band/wrist strap 59. In this way, the apparatus is held in good and intimate contact with respect to the top of the wrist where excellent measurement may be made.

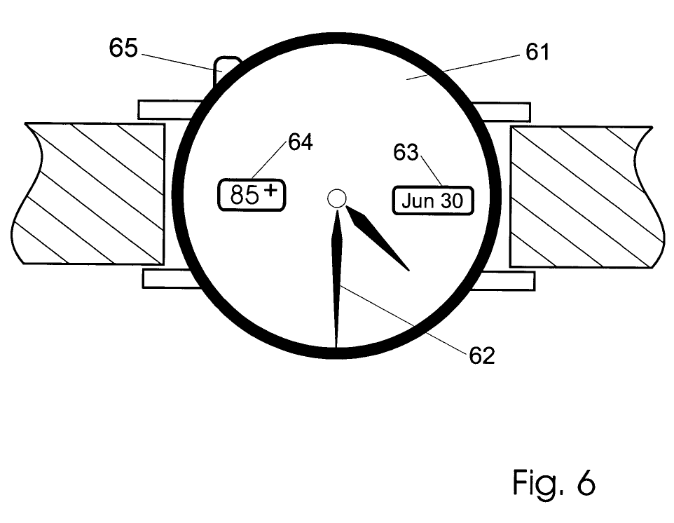

After a successful determination of a blood analyte measurement is made, for example blood glucose concentration, the measurement information is to be passed on in a manner which permits one make good use of the information. In a first example, blood glucose concentration is reported directly to a user in a graphic display at the face of the wrist watch. A wearer can at any time desirable, look to the watch to learn the level in near real-time. FIG. 6 illustrates a top side 61 of a watch case having indication of time of day 62, date 63, and blood glucose level 64. While the drawing suggests a digital display, one might realize a similar function in a mechanical system such as a dial which is popular in wrist watch type instruments. While a display offers continuous presentation of blood glucose level, an alternative includes an audio alarm or digital indicator which becomes activated upon some threshold level.

Because blood glucose levels are important in trend analysis, it is sometimes useful to collect data over long periods and to make that available to analysis at a later time. Accordingly, these devices may include memory facility to record data collected at a plurality of instances and later reported as a time series. In best versions, the device has a data output port 65, a standard electrical connector such as USB connector for example.

The examples above are directed to specific embodiments which illustrate preferred versions of devices and methods of these inventions. In the interests of completeness, a more general description of devices and the elements of which they are comprised as well as methods and the steps of which they are comprised is presented here following.

### Apparatus Of These Inventions

In most general terms, apparatus of these inventions may precisely be described as portable, non-invasive systems for blood analyte measurement integrated with a common article worn about the body. These device primarily include: a rigid case for containing a processing unit in communication with an optical source; and an acoustic detector. The case has a top side and a bottom side. The bottom side is suitable for supporting the acoustic detector and optical source such that they are coupled to human tissue. The elements are arranged to effect a photoacoustic effect measurement of blood analytes via optical stimulation of pressure waves in tissue. Optical sources are comprised of at least one quantum cascade semiconductor laser designed lasing in the Mid-IR spectral range. The optical source may be arranged to operate on a plurality of wavelengths tuned to natural resonances of a glucose molecule. The acoustic detectors are characterized as audio microphones and may be PZT crystal or crystals. The detector can be an array of discrete elements to facilitate tuning to a particular location.

One will now fully appreciate how advanced biometric devices may be integrated with common articles which may be worn on the body. Although the present invention has been described in considerable detail with clear and concise language and with reference to certain preferred versions thereof including the best mode anticipated by the inventor, other versions are possible. Therefore, the spirit and scope of the invention should not be limited by the description of the preferred versions contained therein, but rather by the claims appended hereto

1) Portable, non-invasive systems for blood analyte measurement integrated with a common article wearable about the body comprising:
a rigid case operable for containing;
a processing unit in communication with;
an optical source; and
an acoustic detector,
said rigid case having a top side and a bottom side, the bottom side suitable for supporting said acoustic detector and said optical source whereby they may be coupled to human tissue.

2) Apparatus of claim 1, said rigid case forming an enclosed space of between one and ten cubic centimeters such that the article may be easily worn about the body.

3) Apparatus of claim 2, said elements being integrated together with timekeeping facility to form a wrist watch type article suitable for wear at the wrist.

4) Apparatus of claim 3, said elements are arranged to effect a photoacoustic effect measurement of blood analytes via optical stimulation of pressure waves in an elastic tissue medium.

5) Apparatus of claim 3, said optical source is comprised of at least one quantum cascade semiconductor laser operable for producing beams of light in a Mid-IR spectra between 2-200 microns.

6) Apparatus of claim 5, said beam of light is comprised of a plurality of center wavelengths at least one tuned to natural resonances of a glucose molecule.

7) Apparatus of claim 3, said acoustic detector is characterized as an audio microphone.

8) Apparatus of claim 7, said acoustic detector is at least one PZT crystal operable for producing an electrical output in response to incident pressure.

9) Apparatus of claim 8, said acoustic detector includes an array of discrete elements tuned to a particular region.

10) Apparatus of claim 3, said rigid case bottom side supports an optical and acoustic coupling to tissue and may be fastened to the human wrist whereby the bottom side makes intimate contact with tissue at the top of the wearer's wrist.

11) Apparatus of claim 10, back of watch case provides optical coupling to tissue whereby light penetrates the tissue up to about 100 microns in depth.

12) Apparatus of claim 10, back of watch case provides acoustic coupling to tissue whereby acoustic energy originating in tissue below the skin surface is coupled into the case via said acoustic detector.

13) Apparatus of claim 10, said case further comprises a user interface operable for presenting indicia at a display means.

14) Apparatus of claim 10, said case further comprises a data output port whereby information contained in a memory may be electronically passed to external systems including a computer.
